(19) United States
(12) Patent Application Publication
(10) Pub. No.: US 2007/0042475 A1
(43) Pub. Date: Feb. 22, 2007

(54) Method for the enzymatic production of chiral 1-acylated 1,2-diols

(75) Inventors: Rupert Pfaller, Munich (DE); Anneliese Reutter-Maier, Kirchseeon (DE); Elida Schmid, Wolfratschausen (DE)

Correspondence Address: BROOKS KUSHMAN P.C., 1000 TOWN CENTER, TWENTY-SECOND FLOOR, SOUTHFIELD, MI, 48075, US

(73) Assignee: Consortium fuer Elektrochemische Industrie GmbH, Munich (DE)

(21) Appl. No.: 11/504,288

(22) Filed: Aug. 15, 2006

(30) Foreign Application Priority Data
Aug. 16, 2005 (DE) 10 2005 038 606.7

(51) Int. Cl. C12P 7/62; C12N 9/02

(52) U.S. Cl. 435/135; 435/189

(57) ABSTRACT

A method for the production of a chiral compound of the formula (I), 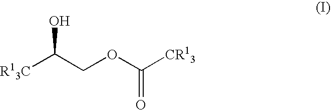
R¹ being identical or different and being H or an organic radical, where a biotransformation composition comprising a compound of the formula (II), 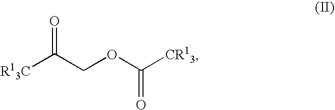 an oxidoreductase, a redox cofactor and a cosubstrate are reacted forming the chiral compound of the formula (I) which is subsequently isolated.

### BACKGROUND OF THE INVENTION

1. Field of the Invention

The invention relates to a method for the enzymatic production of a chiral 1-acylated derivative of a 1,2-diol of the general formula (I), 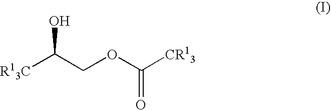
and also of (R)-1-acetoxy-2-propanol (R¹—AcP) (R¹═H).

2. Background Art

Optically active hydroxyl compounds such as the chiral compounds of the general formula (I) are valuable synthesis building blocks, e.g. in the production of pharmaceutical active ingredients or agrochemicals. These compounds can only be produced with difficulty by classical chemical methods, since the required optical purities for applications in the pharmaceutical or agrochemical sector can be achieved only with difficulty in this route. Therefore, for the production of chiral compounds, use of biotechnological methods is increasing. In particular enzymes which can reduce carbonyl compounds are of increasing importance due to their high enantioselectivity.

Compounds of the general formula (I) are of synthetic interest, since only the OH group in the 1-position is substituted, and the OH group in the optically active 2-position is available for further derivatization. Alternative methods for production of these compounds, e.g. direct acylation of 1,2-diols, lead to compounds preferably substituted in the 2-position.

Enzymes of the class of oxidoreductases which are used for production of chiral compounds by reduction of prochiral carbonyl compounds are referred to by the collective name carbonyl reductase (hereinafter “CR”). In the majority of cases the product of a CR reaction is an alcohol. However, it is also possible that the product of a CR reaction is an amine. Carbonyl reductases comprise, inter alia, alcohol dehydrogenases (hereinafter “ADH”), aldo-keto reductases (“AKR”), aldehyde reductases, glycerol dehydrogenases and fatty acid synthetase (called “FAS”). However, carbonyl reductases also comprise amino transferases or amino acid dehydrogenases (e.g. threonine dehydrogenase). This broad spectrum of reducing enzymes have in common the fact that they obtain the electrons for reduction of the carbonyl compound from redox cofactors in their reduced form, customarily NADH or NADPH.

The redox cofactors NADH and NADPH are consumed stoichiometrically in the enzymatic reduction, i.e. they must either be used stoichiometrically or else regenerated by oxidation of a cosubstrate (cofactor regeneration). A cosubstrate here is defined as a compound which is enzymatically oxidized as reducing agent, the electrons produced being transferred to NAD or NADP and NADH or NADPH being thereby regenerated.

For (S)-1-acetoxy-2-propanol (S—AcP), a synthesis has been described by biotransformation using cells or isolated enzymes from baker's yeast (Ishihara et al., BULL. CHEM. SOC. JPN 67: 3314-3319 (1994); Ishihara et al., TETRAHEDRON LETT. 35, 4569, 4570, (1994); and Ishihara et al., J. FERMENT. BIOENG. 3: 266-268 (1996)). The low space-time yields, a complex procedure involving substrate feeding, and use of a high amount of yeast cells, dictate that the use of yeast cells for synthesizing S—AcP on an industrial scale is not practical. Example 7 describes as a comparative example alternative production of S—AcP by the enzyme T-ADH, an S-selective ADH. In this case also, the space-time yields are comparatively low, so that for those skilled in the art it is not possible to derive from the known methods for the production of S—AcP an analogous method using an R selective enzyme having the high space-time yields required for an inexpensive production of R—AcP.

### SUMMARY OF THE INVENTION

It was therefore an object of the invention to provide an enzymatic method which makes possible efficient inexpensive production of chiral compounds of the formula (I). This and other objects were achieved by the biotransformation method disclosed herein, where a biotransformation composition comprising as starting material a compound of the formula (II), 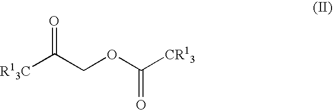
R being identical or different and being H or an organic radical, an oxidoreductase, a redox cofactor and a cosubstrate are allowed to react, a chiral compound of the formula (I) 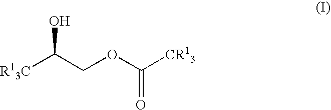
being formed and subsequently isolated.

### DETAILED DESCRIPTION OF THE PREFERRED EMBODIMENT(S)

Preferably, R¹ is identical or different and is H or C₁₋₂₀ alkyl, C₂₋₂₀ alkenyl, C₃₋₈ cycloalkyl, C₆₋₂₀ aryl or C₅₋₂₀ heteroaryl radical, where one or more carbon atoms can be replaced by atoms selected from the group B, N, O, Si, P and S, or where one or more carbon atoms can be substituted by F, Cl, Br, I, C₃₋₈ cycloalkyl, C₆₋₂₀ aryl, C₅₋₂₀ heteroaryl, CN, NH₂, NO or NO₂.

Most preferably, R¹ is H or a C₁₋₂₀ alkyl, C₃₋₈ cycloalkyl, C₆₋₂₀ aryl or C₅₋₂₀ heteroaryl radical, where one or more carbon atoms can be substituted by F, Cl, C₃₋₈ cycloalkyl, C₆₋₂₀ aryl or C₅₋₂₀ heteroaryl. Such compounds are disclosed, for example, in Ishihara et al. (1994), BULL. CHEM. SOC. JPN 67: 3314-3319. With particular preference, R¹ is H.

The oxidoreductase is preferably a CR which has R specificity.

The redox cofactor is a compound which, in its reduced form, provides electrons which, in the enzymatic reaction, are transferred by an oxidoreductase to the starting material with the result that an inventive product is formed. The redox cofactor is preferably selected from compounds of the group NAD, NADP (in each case the oxidized form of the cofactor), NADH, NADPH (in each case the reduced form of the cofactor) and salts thereof.

The redox cofactors in their reduced form, NADH and NADPH, are consumed stoichiometrically in the enzymatic reduction, i.e. they must either be used stoichiometrically or be regenerated by oxidation of a cosubstrate (cofactor regeneration). Stoichiometric use of NADH or NADPH is not economical because of the high price of these compounds. This disadvantage is circumvented by cofactor regeneration. Prerequirements for this are an inexpensive cosubstrate (reducing agent) and a cofactor-reducing enzyme. Industrial use of biocatalytic reduction methods is only made possible by the efficient and cost-effective regeneration of the redox cofactor.

A cosubstrate is a compound which is enzymatically oxidized as reducing agent, the resultant electrons being transferred to NAD or NADP and NADH or NADPH, respectively, being thereby regenerated.

If in the inventive method a CR of the class of ADHs is used, the cosubstrate used for cofactor regeneration is an alcohol, preferably a low cost alcohol such as isopropanol or 2-butanol. However, all other higher secondary alcohols derived from 2-butanol are also suitable. Thus in this method variant not only the stereoselective reduction of the starting material but also cofactor regeneration of the same enzyme, ADH, is ensured.

If use is made of a CR which is not an ADH in the inventive method, cofactor regeneration is performed by means of a second enzyme, likewise situated in the reaction mixture. The CR reduces the starting material stereoselectively to the desired product, the cofactor NADH or NADPH being consumed. The consumed NADH or NADPH is regenerated by a second enzyme. In principle any enzyme for cofactor regeneration is suitable which oxidizes a substrate in an enzymatic reaction and simultaneously reduces NAD to NADH or NADP to NADPH. Preferably, use is made of an enzyme which oxidizes a cosubstrate which is as inexpensive as possible, for example glucose, formic acid, or salts thereof. Preferably, as an enzyme for cofactor regeneration, use is made of an enzyme from the glucose dehydrogenase (GDH) and formate dehydrogenase (FDH) groups.

Preferred combinations of enzyme/cosubstrate for cofactor regeneration are the combination of an ADH with an alcohol such as isopropanol or 2-butanol, or the combination of a GDH with glucose. Particular preference is given to the combination of an ADH with an alcohol, such as e.g. isopropanol or 2-butanol, and in particular, preference is given to the combination of an ADH with isopropanol.

The inventive method makes it possible, by enzymatic reduction of a starting material of the formula (II), to produce compounds of the formula (I) at high space-time yields with low amounts of enzyme by means of a simple batch method.

Starting materials of the general formula (II) can be produced according to the prior art, e.g. by reaction of 1-chloroketones, particularly 1-chloroacetone, with the salt of a carboxylic acid. Thus the particularly preferred starting material acetoxyacetone can be produced in this manner from 1 chloroacetone and potassium acetate or sodium acetate (Ishihara et al., BULL. CHEM. SOC. JPN 67: 3314-3319 (1994)). However, any other salt of acetic acid is also suitable for the synthesis of acetoxyacetone. Particular preference is given to the production of acetoxyacetone from 1-chloroacetone and sodium acetate by a continuous method.

As R-specific CRs, use is preferably made of secondary ADHs, e.g. from strains of the genus Lactobacillus such as the ADHs from Lactobacillus brevis (LB-ADH), Lactobacillus kefir, Lactobacillus parabuchneri, Lactobacillus kandleri, Lactobacillus minor, or use is made of fatty acid synthetases (FAS), most preferably the FAS of baker's yeast or from Pichia pastoris. Preferred R-selective CRs are ADHs of the genus Lactobacillus. A most preferred R-selective CR is LB-ADH.

The CRs used for the enzymatic reduction can be produced by culturing the microorganism from which the CR in question originates. This is performed in each case in a manner known to those skilled in the art. The CR enzyme produced in this manner can be used directly in the cells of the production host, but it can also be used after digestion of the cells as a protein extract, or used as purified protein after appropriate workup, e.g. by column chromatography.

The CR enzyme production can be performed using an expression system, also in recombinant form. For this the gene coding for the CR in question is isolated and, in accordance with the prior art, cloned into an expression vector suitable for the protein production. After transformation of the expression vector into a suitable host organism, a production strain is isolated. Using this production strain the CR may be produced in a manner known per se, e.g. by fermentation. The CR enzyme produced in this manner can be further used directly in the cells of the production host, as protein extract after digestion of the cells, or as purified protein after appropriate workup, e.g. by column chromatography. Preference is given to enzyme production of the inventive CRs using an expression system in recombinant form.

Bacterial and eukaryotic expression systems are suitable for enzyme production. Host organisms for enzyme production are preferably selected from Escherichia coli, strains of the genus Bacillus, yeasts such as Pichia pastoris, Hansenula polymorpha or Saccharomyces cerevisiae, and also fungi such as Aspergillus or Neurospora, but they are not restricted to these host organisms. The preferred expression systems comprise E. coli, Bacillus, Pichia pastoris, S. cerevisiae, Hansenula polymorpha or Aspergillus, and particularly preferred expression systems for production of the CR enzyme are E. coli, Pichia pastoris and S. cerevisiae. An expression system preferred in particular is E. coli.

To achieve enzyme usage as cost-efficient as possible, the enzyme production is preferably performed by fermentation, most preferably in a fed batch method.

Preferably, the cells from the fermentation are then further used directly, suspended in the fermentation medium (fermentor cells) or after prior isolation and subsequent resuspension so that the method is performed as whole cell biotransformation. However, it is also possible to make use of the resultant protein extract after digestion of the cells, or the resultant purified protein after appropriate workup, e.g. by column chromatography.

Particular preference is given to whole cell biotransformation in which firstly the enzyme production proceeds in a recombinant host cell by means of fermentation and the fermentor cells are subsequently directly used, suspended in fermentation medium, in an inventive biotransformation.

In its simplest form, an inventive biotransformation composition comprises (what are termed “batch solution”) fermentor cells containing a CR enzyme, a compound of the formula (II) as starting material; a redox cofactor selected from the compounds NAD, NADH, NADP, NADPH, and salts thereof; a cosubstrate selected from isopropanol, 2-butanol and glucose, and when glucose is used as cosubstrate, a GDH as cofactor-regenerating enzyme.

In a modified form of the method, one or more of the components of the biotransformation composition are added continuously or batchwise (termed the fed-batch procedure). The preferred method is the batch procedure.

Preferably, an inventive biotransformation composition contains between 1% (v/v) and 40% (v/v) of a suspension of fermentor cells obtained from the fermentation having a biomass fraction of 0.05-2% (w/v), containing a CR enzyme. The biomass fraction is defined here as dry biomass which is obtained when the fermentor cells are dried to constant weight, e.g. in a drying cabinet at 105° C. More preferably, the composition contains between 5% (v/v) and 30% (v/v) of a suspension of fermentor cells obtained from the fermentation containing a CR enzyme having a biomass fraction of 0.25-1.5% (w/v). In particular, the composition contains between 10% (v/v) and 25% (v/v) of a suspension of fermentor cells obtained from the fermentation containing a CR enzyme having a biomass fraction of 0.5-1.25% (w/v).

An inventive biotransformation composition is further distinguished in that the fraction of starting material of the formula (II) is between 10% (w/v) and 60% (w/v) of the total batch, preferably between 20% (w/v) and 50% (w/v) of the total batch, and in particular between 30% (w/v) and 45% (w/v) of the total batch.

An inventive biotransformation composition is also distinguished by the fact that the fraction of cosubstrate in the case of isopropanol or 2-butanol is preferably between 10% (w/v) and 50% (w/v) of the total batch, more preferably between 20% (w/v) and 45% (w/v) of the total batch, and most preferably, between 30% (w/v) and 40% (w/v) of the total batch. If glucose is used as cosubstrate, the composition preferably contains glucose preferably in a concentration of 20% (w/v) to 65% (w/v) based on the total batch.

The biotransformation composition preferably comprises the redox cofactor in a concentration between 10 μM and 200 μM, more preferably between 20 μM and 150 μM, and most preferably between 40 μM and 100 μM.

The inventive method is preferably carried out at a temperature of 3° C. to 70° C., more preferably from 5° C. to 50° C., and most preferably from 15° C. to 40° C.

Preferably the method is carried out in a pH range from 5 to 9, more preferably from 5.5 to 8, most preferably from 6 to 7.5. Preferably, the batch is buffered to maintain the pH. Control of pH control is preferably performed via a titration apparatus coupled to a pH meter (the pH stat method).

The reaction time is preferably 5 h to 100 h, more preferably 10 h to 60 h, and most preferably 15 h to 40 h.

Under the foregoing conditions, starting materials of the general formula (II) are preferably converted to a product of the general formula (I) in a yield of >80%, more preferably >90%, and most preferably >93%.

The inventive method has made it possible, for the first time, to produce compounds of the general formula (I), in particular R¹—AcP. In particular it has been surprisingly discovered that starting materials of the general formula (II), preferably acetoxyacetone, in a concentration of 20-43% (w/v), can be converted over a reaction time of 24 h into a product of the general formula (I) at a conversion rate of more than 90%, at an enantiomeric purity ee of 100%, while at the same time the concentration of CR-containing cells, expressed as dry biomass of fermentor cells, is no more than 1% (w/v) of the batch volume. The surprising efficiency and high space-time yields were unexpected from the prior art.

The product may be extracted according to methods known per se, preferably using a water-immiscible organic solvent. The extraction can proceed batchwise or continuously. As is known to those skilled in the art, a temperature is established which ensures optimum extraction of the product from the aqueous phase. Preferably, the extraction proceeds at a temperature of 10° C. to 70° C. Alternatively, direct product isolation by distillation is also possible.

Suitable organic solvents are all water-immiscible solvents which can extract a compound of the formula (I) from an aqueous phase. Preferably, use is made of organic solvents selected from the group of esters, ethers, alkanes and aromatics, most preferably, ethyl acetate, methyl acetate, propyl acetate, isopropyl acetate, butyl acetate, tert-butyl acetate, diethyl ether, diisopropyl ether, dibutyl ether and methyl tert-butyl ether (MTBE), pentane, hexane, heptane, toluene or mixtures thereof. Solvents preferred in particular are MTBE, ethyl acetate and butyl acetate.

The separated organic extraction phase, is preferably worked up by distillation, enrichment of the reaction product being thereby achieved, and partial to complete removal of byproducts from the extraction solvent being effected concurrently. The solvent is able to be used again for the extraction.

By purifying the organic extraction solution containing the crude product, for example by means of fine distillation, the desired end product is obtained. The end product is typically obtained in a yield >70%, preferably >80%, and more preferably >90%, in each case based on the amount of starting material (II) used. The end product has an enantiomeric excess preferably of ee >90%, more preferably ee >97%, in particular more ee=100%.

### EXAMPLE 1

### Production of LB-ADH by Fermentation

The enzyme LB-ADH, its gene, and the recombinant production of LB-ADH in E. coli are disclosed in EP796914. The plasmid pADH-1 transformed into E. coli and disclosed in EP796914 was used. Alternatively, the enzyme can be obtained commercially from Jülich Fine Chemicals GmbH as crude extract produced from recombinant E. coli.

Fermentation of LB-ADH-Producing E. coli:

Production of an Inoculum for the Fermentation:

1st preculture of E. coli pADH-1 in LBamp medium. Culture was performed for 7 to 8 h on an orbital shaker (Infors) at 120 rpm and 30° C. LBamp medium contained peptone vegetable (Oxoid) 10 g/l; yeast extract (Oxoid) 5 g/l; NaCl 5 g/l and ampicillin 0.1 g/l.

2nd preculture: 100 ml of SM3amp medium were inoculated into a 1 l Erlenmeyer flask with 1.3 ml of shake culture. Culture was performed for 16-18 h at 30° C. and 120 rpm on an orbital shaker up to a cell density OD₆₀₀/ml of 7-10. 100 ml of the preculture were used for inoculating 1 l of fermentor medium. SM3amp medium contained peptone vegetable (Oxoid) 5 g/l; yeast extract (Oxoid) 2.5 g/l; NaCl 0.1 g/l; ammonium sulfate 5 g/l; KH₂PO₄ 3 g/l; K₂HPO₄ 12 g/l; glucose 5 g/l; MgSO₄.7 H₂O 0.3 g/l; CaCl₂.2 H₂O 14.7 mg/l; FeSO₄.7 H₂O 2 mg/l; sodium citrate.2 H₂O 1 g/l; vitamin B1 5 mg/l; trace element mix 1 ml/l, and ampicillin 0.1 g/l. The trace element mix had the composition H₃BO₃ 2.5 g/l; CoCl₂.6 H₂O 0.7 g/l; CuSO₄.5 H₂O 0.25 g/l; MnCl₂.4 H₂O 1.6 g/l; ZnSO₄.7 H₂O 0.3 g/l and Na₂MoO₄.2 H₂O 0.15 g/l.

The fermentations were carried out in Biostat CT fermentors from Sartorius BBI Systems GmbH. Fermentation medium was FM2amp. The fermentation was performed in the fed-batch mode. FM2amp medium contained glucose 20 g/l; peptone vegetable (Oxoid) 5 g/l; yeast extract (Oxoid) 2.5 g/l; ammonium sulfate 5 g/l; NaCl 0.5 g/l; FeSO₄.7 H₂O 75 mg/l; Na₃ citrate.2 H₂O 1 g/l; CaCl₂.2 H₂O 14.7 mg/l; MgSO₄.7 H₂O 0.3 g/l; KH₂PO₄ 1.5 g/l; trace element mix 10 ml/l; vitamin B1 5 mg/l and ampicillin 0.1 g/l. The pH of the FM2amp medium was set to 7.0 before the start of fermentation.

1 l of FM2amp was inoculated with 100 ml of inoculum. The fermentation temperature was 30° C. The pH of the fermentation was 7.0 and was kept constant using the correction media 25% NH₄OH or 6 N H₃PO₄. Aeration was performed using compressed air at a constant flow rate of 5 slpm (standard liter per minute). The oxygen partial pressure pO₂ was set to 50% saturation. The oxygen partial pressure was controlled via the stirring speed (stirrer speed 450-1300 rpm). To control foam formation, Struktol J673 (20-25% v/v in water) was used.

In the course of the fermentation, the glucose consumption was determined by off-line glucose measurement using a glucose analyzer from YSI. As soon as the glucose concentration of the fermentation batches was approximately 5 g/l (5-6 h after inoculation), addition of a 60% w/w glucose feed solution was started. The flow rate of the feed was chosen in such a manner that a glucose concentration of 1-5 g/l could be maintained during the production phase.

LB-ADH production was induced by addition of IPTG (stock solution 100 mM) in a concentration of 0.4-0.8 mM, as soon as the cell growth in the fermentor had reached an OD₆₀₀/ml of 50-60. The total fermentation period was 32 h. After termination of the fermentation, the fermentor broth (dry biomass 50 g/l) was frozen in aliquots each of 100 ml.

### EXAMPLE 2

### Production of an LB-ADH Crude Extract

2 l of cell suspension from the fermentation of E. coli pADH-1 (see Example 1) were centrifuged (15 min 8000 rpm at 4° C., GS 3 rotor, Sorvall centrifuge). The sediment was resuspended in 500 ml of 50 mM potassium phosphate, pH 7.0, 1 mM MgCl₂ and digested by three passages through a high-pressure homogenizer (NS1001L Panda 2K from Niro Soavi) at 800 bar pressure. The homogenate was centrifuged (30 min 8000 rpm at 4° C., GS 3 rotor, Sorvall centrifuge). The supernatant produced an LB-ADH crude extract of 535 ml volume. The LB-ADH activity determination found a volume activity of 1300 U/ml, and a specific activity of 108 U/mg of protein in the crude extract.

Spectrophotometric Determination of LB-ADH Activity:

The assay batch of 1 ml volume for the photometric determination of LB-ADH activity was composed of assay buffer (0.1 M potassium phosphate, pH 7.0, 0.1 M NaCl, 1 mM MgCl₂), 3 μl of substrate ethyl 4-chloroacetoacetate, 0.2 mM NADPH and LB ADH-containing cell extract. Assay temperature was 25° C. The reaction was started by addition of the LB-ADH cell extract and the decrease in extinction owing to consumption of NADPH was measured at a wavelength of 340 nm (extinction coefficient of NADPH: ε=0.63×10⁴ l mol⁻¹×cm⁻¹). One unit of LB-ADH activity is defined as the consumption of 1 μmol of NADPH/min under test conditions.

For determination of the specific activity, the protein concentration of the cell extracts was determined in a manner known per se using the “BioRad Protein Assay” from BioRad.

### EXAMPLE 3

### Gas-Chromatographic Analysis

GC-MS Analysis:

For production of a racemic reference substance of 1-acetoxy-2-propanol, acetoxyacetone (CAS:RN 529-20-1) was unselectively reduced in a manner known per se by treatment with NaBH4. The product mixture, which in addition to the main product 1-acetoxy-2-propanol also contained in small amounts the rearrangement product 2-acetoxy-1-propanol, was analyzed by GC-MS, without chiral separation according to the prior art. The molar masses determined at the following retention times were:
acetoxyacetone: 3.71 min, molar mass 116.
1-acetoxy-2-propanol: 3.97 min, molar mass 118.
2-acetoxy-1-propanol: 4.17 min, molar mass 118.
   Chiral GC:

Use was made of a gas chromatograph 6890N from Agilent including flame-ionization detector, which was equipped with a CP-Chirasil-Dex-CB column from Varian (25 m×0.25 mm) for chiral separation.

For the gas-chromatographic separation, a temperature gradient of 100° C.-140° C. was set with a gradient slope of 2° C./min, followed by a temperature gradient of 140° C.-170° C. with a gradient slope of 10° C./min. Retention times under these conditions were:
acetoxyacetone: 4.4 min.
(R)-1-acetoxy-2-propanol: 6.2 min.
(S)-1-acetoxy-2-propanol: 6.4 min.
(R)-2-acetoxy-1-propanol: 7.8 min.
(S)-2-acetoxy-1-propanol: 8.7 min.

### EXAMPLE 4

### Product Detection by Saponification of (R)-1-acetoxy-2-propanol to Give the (R)-Propylene Glycol and Analysis by Chiral GC

100 ml of reaction mixture from a batch as described in Example 5 were extracted three times, each with 100 ml of MTBE and the solvent was distilled off from the combined extraction phases in a rotary evaporator.

The resultant crude product of (R)-1-acetoxy-2-propanol was saponified by treatment with NaOH. The batch, for saponification, contained 1 ml of crude product, 1.5 ml of H₂O, 1 ml of 10 M NaOH and 7.5 ml of methanol. After 2 h of incubation at 50° C., 1 ml of the batch was extracted using 1 ml of MTBE and resultant propylene glycol was analyzed by chiral GC, employing gas chromatograph 6890N from Agilent with a flame-ionization detector, and equipped with a CP-Chirasil-Dex-CB column from Varian (25 m×0.25 mm) for chiral separation.

For the gas-chromatographic separation, a temperature gradient of 65° C.-170° C. having a gradient slope of 15° C./min was set. Reference substances for (R)- and (S)-propylene glycol are commercially available (Sigma Aldrich). Under the conditions of chiral GC, the following retention times were found:
(S)-propylene glycol: 18.1 min.
(R)-propylene glycol: 18.4 min.

The (R)-propylene glycol obtained from the biotransformation after saponification appeared as a single peak. Spiking experiments using (R)- and (S)-propylene glycol reference substances confirmed that the biotransformation of acetoxyacetone led selectively to (R)-1-acetoxy-2-propanol. The byproduct of the (S)-enantiomer could not be detected. The enantiomeric excess ee was 100%.

In an analysis carried out similarly where the crude product had been obtained from the biotransformation using T-ADH (Example 7), correspondingly (S)-propylene glycol having an ee of 100% was detected.

### EXAMPLE 5

### Biotransformations Using LB-ADH Cells

20% acetoxyacetone concentration: a reaction batch was composed of 20 ml (21.5 g) of acetoxyacetone, 40 ml (31.4 g) of isopropanol, 12 ml of LB-ADH cells (fermentor broth as described in the 1st Example), 50 μM NADP and 28 ml of KPi buffer. The composition of KPi buffer was 0.1 M potassium phosphate, pH 7.0, 0.1 M NaCl, 1 μM MgCl₂. The reaction batch was stirred at 30° C. At various time points, 0.1 ml samples of the reaction batch were taken, extracted with 1 ml of MTBE and analyzed by chiral GC (see Example 3). After 24 h the reaction conversion rate of the acetoxyacetone used was 99%. The enantiomeric excess ee of the product (R)-1-acetoxy-2-propanol was 100%.

30% acetoxyacetone concentration: a reaction batch was composed of 30 ml (32.3 g) of acetoxyacetone, 40 ml (31.4 g) of isopropanol, 12 ml of LB-ADH cells, 50 μM NADP and 18 ml of KPi buffer. The reaction batch was stirred at 30° C. At various time points 0.1 ml samples of the reaction batch were taken, extracted with 1 ml of MTBE and analyzed by chiral GC (see Example 3). After 24 h the reaction conversion rate of the acetoxyacetone used was 95%. The enantiomeric excess ee of the product (R)-1-acetoxy-2-propanol was 100%.

40% acetoxyacetone concentration: a reaction batch was composed of 40 ml (43 g) of acetoxyacetone, 40 ml (31.4 g) of isopropanol, 12 ml of LB-ADH cells, 50 μM NADP and 8 ml of KPi buffer. The reaction batch was stirred at 30° C. At various time points, 0.1 ml samples of the reaction batch were taken, extracted with 1 ml of MTBE and analyzed by chiral GC (see Example 3). After 24 h the reaction conversion rate of the acetoxyacetone used was 89%. The enantiomeric excess ee of the product (R)-1-acetoxy-2-propanol was 100%.

40% acetoxyacetone dosage: a reaction batch was composed of 40 ml (43 g) of acetoxyacetone, 40 ml (31.4 g) of isopropanol, 20 ml of LB-ADH cells and 50 μM NADP. pH of the reaction was 7.0. The reaction batch was stirred at 30° C. At various time points, 0.1 ml samples of the reaction batch were taken, extracted with 1 ml of MTBE and analyzed by chiral GC (see 3rd Example). After 24 h, the reaction conversion rate of the acetoxyacetone used was 95%. The enantiomeric excess ee of the product (R)-1-acetoxy-2-propanol was 100%.

### EXAMPLE 6

### Biotransformation Using LB-ADH Crude Extract

30% acetoxyacetone concentration: a reaction batch was composed of 30 ml (32.3 g) of acetoxyacetone, 40 ml (31.4 g) of isopropanol, 30 ml of extract of LB-ADH cells (39,000 U LB-ADH, see 2nd Example) and 50 μM NADP. The reaction batch was stirred at 30° C. At various time points, 0.1 ml samples of the reaction batch were taken, extracted with 1 ml of MTBE and analyzed by chiral GC (see Example 3). After 24 h, the reaction conversion rate of the acetoxyacetone used was 90%. The enantiomeric excess ee of the product (R)-1-acetoxy-2-propanol was 100%.

### EXAMPLE 7 (COMPARATIVE EXAMPLE)

### Biotransformation Using T-ADH Enzyme

10% acetoxyacetone concentration: a reaction batch was composed of 10 ml (10.8 g) of acetoxyacetone, 20 ml (15.7 g) of isopropanol, 40 ml of KPi buffer, pH 7.0, 30 ml of T-ADH enzyme (6000 U of T-ADH, obtained from Jülich Fine Chemicals GmbH) and 50 μM NADP. The reaction batch was stirred at 30° C. At various time points, 0.1 ml samples of the reaction batch were taken, extracted with 1 ml of MTBE and analyzed by chiral GC (see Example 3). After 24 h, the reaction conversion rate of the acetoxyacetone used was 91%. The enantiomeric excess ee of the product (S)-1-acetoxy-2-propanol was 100%.

20% acetoxyacetone concentration: a reaction batch was composed of 20 ml (21.5 g) of acetoxyacetone, 30 ml (23.6 g) of isopropanol, 5 ml of KPi buffer, pH 7.0, 45 ml of T-ADH enzyme (9000 U of T-ADH, obtained from Jülich Fine Chemicals GmbH) and 50 μM NADP. The reaction batch was stirred at 30° C. At various time points, 0.1 ml samples of the reaction batch were taken, extracted with 1 ml of MTBE and analyzed by chiral GC (see Example 3). After 24 h, the reaction conversion rate of the acetoxyacetone used was 36.3%. The enantiomeric excess ee of the product (S)-1-acetoxy-2-propanol was 100%.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method for the production of a chiral compound of the formula (I), 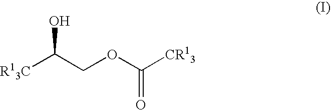
R¹ being identical or different and being H or an organic radical, where a biotransformation composition comprising a compound of the formula (II), 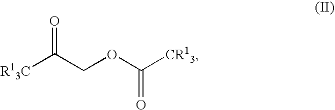
an oxidoreductase, a redox cofactor, and a cosubstrate are reacted to form the chiral compound (I), and isolating the chiral compound (I).

2. The method of claim 1, wherein R¹ is identical or different and is H or C₁₋₂₀ alkyl, C₂₋₂₀ alkenyl, C₃₋₈ cycloalkyl, C₆₋₂₀ aryl or C₅₋₂₀ heteroaryl, where one or more carbon atoms can be replaced by atoms selected from the group B, N, O, Si, P and S, and where one or more carbon atoms can be substituted by F, Cl, Br, I, C₃₋₈ cycloalkyl, C₆₋₂₀ aryl, C₅₋₂₀ heteroaryl, CN, NH₂, NO or NO₂.

3. The method of claim 1, wherein R¹ is identical or different and is H or C₁₋₂₀ alkyl, C₃₋₈ cycloalkyl, C₆₋₂₀ aryl or C₅₋₂₀ heteroaryl, where one or more carbon atoms can be substituted by F, Cl, C₃₋₈ cycloalkyl, C₆₋₂₀ aryl or C₅₋₂₀ heteroaryl.

4. The method of claim 1, wherein R¹ is H.

5. The method of claim 1, wherein the oxidoreductase is a carbonyl reductase having R-specificity.

6. The method of claim 5, wherein the R-specific carbonyl reductase comprises a secondary ADH or a fatty acid synthetase.

7. The method of claim 5, wherein the R-specific carbonyl reductase comprises an LB-ADH from Lactobacillus brevis.

8. The method of claim 1, wherein at least one redox cofactor is a compound selected from the group consisting of NAD, NADP, NADH, NADPH, and salts thereof.

9. The method of claim 1, wherein the cosubstrate is a compound which is enzymatically oxidized as reducing agent, electrons being transferred to NAD thereby generating NADH, or to NADP thereby generating NADPH.

10. The method of claim 1, wherein the carbonyl reductase is an alcohol dehydrogenase and the cosubstrate is an alcohol.

11. The method of claim 10, wherein the alcohol is isopropanol or 2-butanol.

12. The method of claim 1, wherein starting materials of the general formula (II) are converted to a compound of the formula (I) with a conversion efficiency >80%.

13. The method of claim 1, wherein starting materials of the general formula (II) are converted to a compound of the formula (I) with a conversion efficiency >90%.

14. The method of claim 1, wherein the compound of the formula (I) is extracted from the reaction batch by means of a water-immiscible organic solvent, or is separated by distillation.

15. A biotransformation composition suitable for use in the method of claim 1, comprising fermentor cells containing a CR enzyme; a compound of the formula (II); at least one redox cofactor selected from the group consisting of NAD, NADH, NADP, NADPH, and salts thereof; at least one cosubstrate selected from the group consisting of isopropanol, 2-butanol and glucose; and when glucose is a cosubstrate, a GDH cofactor-regenerating enzyme.

16. The composition of claim 15 containing between 1% (v/v) and 40% (v/v), based on the total composition, of fermentation medium having fermentor cells having a biomass fraction of 0.05-2% (w/v) containing a CR enzyme; a compound of the formula (II) in an amount of 10% (w/v) to 60% (w/v) of the total batch; and between 10% (w/v) and 50% (w/v), based on the total batch, of a cosubstrate selected from the group isopropanol and 2-butanol; and a redox cofactor in an amount between 10 μM and 200 μM.

17. The composition of claim 15, wherein the CR enzyme is an ADH.

18. The composition of claim 15, wherein the compound of the formula (II) is acetoxyacetone (R¹═H).

19. A compound of the general formula (I), R¹ being H ((R)-1-acetoxy-2-propanol).
